(19) United States
(12) Patent Application Publication
(10) Pub. No.: US 2026/0213020 A1
(43) Pub. Date: Jul. 23, 2026

(54) TRAINING DEVICE, TRAINING METHOD, AND RECORDING MEDIUM

(71) Applicant: NEC Corporation, Tokyo (JP)

(72) Inventors: Chenhui HUANG, Tokyo (JP); Kensuke WAGATA, Tokyo (JP); Fumiyuki NIHEY, Tokyo (JP)

(73) Assignee: NEC Corporation, Tokyo (JP)

(21) Appl. No.: 19/452,935

(22) Filed: Jan. 20, 2026

(30) Foreign Application Priority Data
Jan. 23, 2025 (JP) 2025-009871

(51) Int. Cl. G16H 50/30; G06N 20/00

(52) U.S. Cl. CPC G16H 50/30; G06N 20/00

(57) ABSTRACT

In the training device, the estimation unit calculates an estimated value of a response variable from an input explanatory variable. The difference calculation unit calculates the difference between a ground truth of the response variable and the estimated value. The distribution calculation unit calculates a probability density function from the ground truth of the response variable to obtain a probability distribution. The loss calculation unit calculates a loss by weighting the difference by a weight based on the probability distribution. The training unit trains the estimation unit, based on the loss. By using a machine learning model trained by the training device to estimate disease risk, it is possible to support decision-making regarding the lifestyle habits of subjects.

### INCORPORATION BY REFERENCE

This application is based upon and claims the benefit of priority from Japanese Patent Application 2025-009871, filed on Jan. 23, 2025, the disclosure of which is incorporated herein in its entirety by reference.

### TECHNICAL FIELD

The present disclosure relates to a loss function for use in training of a machine learning model.

### BACKGROUND ART

In general, regarding training of a machine learning model, training is performed in such a way as to minimize a loss calculated based on both a prediction value or estimated value output from the model based on an input value, and a ground truth. For example, JP2001-272994A describes a training device that performs training in such a way as to minimize a loss between a class output from a model and a correct answer.

### SUMMARY

In general, a loss function for use in training of a model is designed in such a way as to reduce the sum total of the differences between estimated values and their associated ground truths in the entire distribution of training data. However, for example, for a model for detecting abnormalities in health, data of unhealthy persons is important. However, the number of unhealthy persons to a total sample size of training data is small. Therefore, as a problem, the contribution ratio of data of unhealthy persons to the entire loss is low and the data of unhealthy persons is difficult to effectively use in training of a model.

One object of the present disclosure is to provide a training device enabling training by effectively using data which deviates from the center in a distribution of training data.

According to an example aspect of the present invention, there is provided a training device comprising:
an estimation means for calculating an estimated value of a response variable from an input explanatory variable using an estimation model;
a difference calculation means for calculating a difference between a ground truth of the response variable and the estimated value;
a distribution calculation means for calculating a probability density function from the ground truth of the response variable to obtain a probability distribution;
a loss calculation means for calculating a loss by weighting the difference by a weight based on the probability distribution; and
a training means for training the estimation model, based on the loss.

According to another example aspect of the present invention, there is provided a training method to be performed by a computer, comprising:
calculating an estimated value of a response variable from an input explanatory variable using an estimation model;
calculating a difference between a ground truth of the response variable and the estimated value;
calculating a probability density function from the ground truth of the response variable to obtain a probability distribution;
calculating a loss by weighting the difference by a weight based on the probability distribution; and
training the estimation model, based on the loss.

According to still another example aspect of the present invention, there is provided a non-transitory computer-readable recording medium storing a program, the program causing a computer to perform processing comprising:
calculating an estimated value of a response variable from an input explanatory variable using an estimation model;
calculating a difference between a ground truth of the response variable and the estimated value;
calculating a probability density function from the ground truth of the response variable to obtain a probability distribution;
calculating a loss by weighting the difference by a weight based on the probability distribution; and
training the estimation model, based on the loss.

According to the present disclosure, it is possible to provide a training device enabling training by effectively using data, which deviates from the center in a distribution of training data.

### BRIEF DESCRIPTION OF THE DRAWINGS

FIG. 1 illustrates exemplary loss functions;

FIG. 2 illustrates an exemplary probability distribution of training data;

FIG. 3 is an explanatory graph for hyperparameters in a loss function of a proposed technique;

FIG. 4 illustrates an entire configuration of a training device according to the present disclosure;

FIG. 5 is a block diagram illustrating a hardware configuration of the training device;

FIG. 6 is a block diagram illustrating a functional configuration of the training device;

FIG. 7 is a flowchart of training processing;

FIGS. 8A and 8B illustrate experimental results;

FIG. 9 illustrates an application example of the training device;

FIG. 10 is a block diagram illustrating a configuration of another training device according to the present disclosure; and

FIG. 11 is a flowchart of processing in another training processing.

### EXAMPLE EMBODIMENTS

Preferred example embodiments of the present disclosure will be described below with reference to the drawings.

### DESCRIPTION OF BACKGROUND

Using medical examination data tracked on a time-series basis (hereinafter, referred to as “medical examination data”), a value of medical examination data in the future can be predicted, leading to early detection of any abnormality in health. In general, since the ratio of unhealthy persons to population is small, the values of medical examination data of unhealthy persons deviate from the average value and thus are often expressed as outliers. However, such medical examination data of unhealthy persons is important for prediction of an abnormality in health. Various loss functions available for robust estimation of an abnormality in health using medical examination data have been proposed.

However, the existing loss functions aim to reduce the sum total of the differences between ground truths and estimated values in the entire distribution of training data. Thus, like the above-described medical examination data of unhealthy persons, a small number of pieces of data largely deviating from the center of a distribution have a low contribution ratio to the sum total of losses in the entire distribution and thus tend to be neglected or ignored in training of an estimation model.

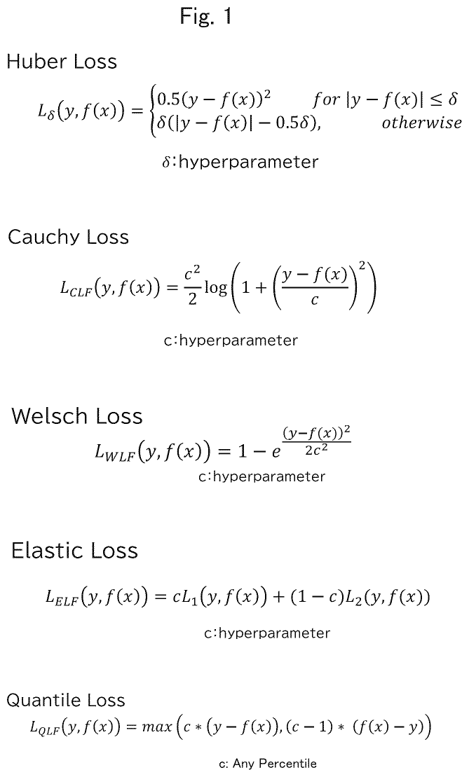

FIG. 1 illustrates some exemplary loss functions. The Huber loss has the advantage of mean squared error (MSE) and the advantage of mean absolute error (MAE) in combination. The Huber loss makes a switch between the mean squared error and the mean absolute error, depending on whether an absolute value |(y−f(x))| of the difference between a ground truth y and a prediction value f(x) is larger than a threshold δ, but does not take a distribution characteristic of training data (response variable) into account.

The Cauchy loss is a robust loss function against outliers and thus is less affected by outliers as a characteristic. This loss function uses the difference between a ground truth and a prediction value, but does not take a distribution characteristic of a response variable into account.

The Welsch loss is also a robust loss function against outliers and suppresses the influence of outliers as a characteristic. This loss function also uses the difference between a ground truth and a prediction value, but does not take a distribution characteristic of a response variable into account.

The Elastic loss is also a robust loss function against outliers. This loss function also uses the difference between a ground truth and a prediction value, but does not take a distribution characteristic of a response variable into account.

The Quantile loss is used to minimize a prediction error to a particular quantile. This loss function divides a distribution based on a freely selected percentile, but enables only two divisions.

Such known loss functions as above are not designed in such a way as to calculate a proper loss in accordance with a distribution of training data.

In this view, the present disclosure proposes a loss function adaptive to a distribution due to weighting to loss using a distribution characteristic of training data. Thus, a robust estimation is enabled with emphasis on outliers, such as the above-described medical examination data of unhealthy persons.

### [Loss Function]

The basic form of a loss function proposed in the present disclosure is expressed by the following Expression (1).

$${L}_{org 1} = {\underbrace{\begin{matrix}\vert y - f ( x ) \vert & \end{matrix}}_{DIFFERENCE}}^{{\overbrace{\begin{matrix}a * ( 1 - {e}^{\frac{- b \vert y - μ \vert}{σ}} ) + c & \end{matrix}}}^{EXPONENT ( POWER )}} = {\vert y - f ( x ) \vert}^{{\overbrace{\begin{matrix}a * ( 1 - {e}^{b \vert z \_ score \vert} ) + c & \end{matrix}}}^{EXPONENT ( POWER )}} \qquad ( 1 )$$

A loss function L_{org1} uses the Minkowski distance and includes a difference and an exponent that exponentiates the difference. The exponent of the Minkowski distance is called a “power”. The power corresponds to a weight in a loss function of a proposed technique. The exponent indicates a deviation (degree of deviation) from the center of a probability distribution of training data, and is defined by an average u and a standard deviation o as parameters of a probability density function indicating a normal distribution. The exponent can be expressed by the following Expression (2) using a distance z_score from the center of the probability distribution of training data. The distance z_score is calculated from the average μ and the standard deviation σ. Note that a, b, and c are hyperparameters.

$$power = a * ( 1 - {e}^{\frac{- b \vert z \_ score \vert}{k}} ) + c \qquad ( 2 )$$

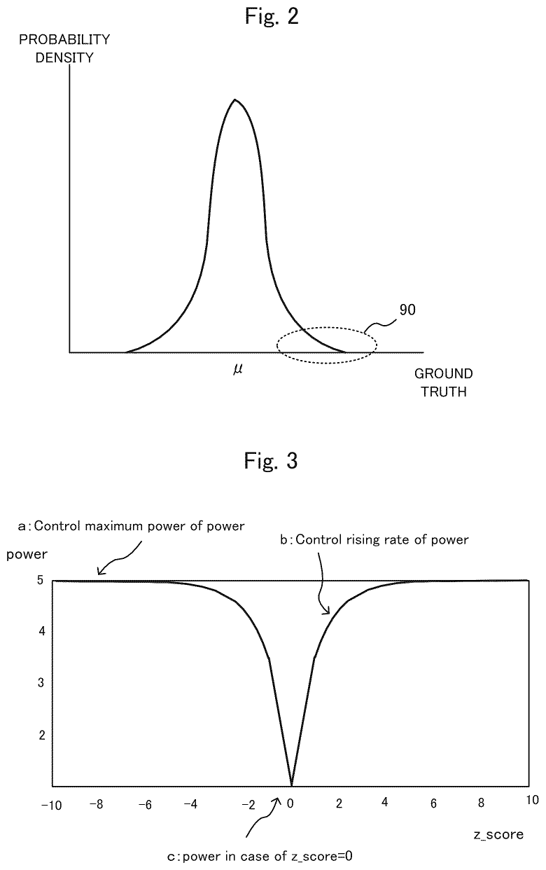

FIG. 2 illustrates an exemplary probability distribution of training data. Data of unhealthy persons in medical examination data gathers in a region 90 deviating from the center of the distribution (hereinafter, also referred to as an “outlier region”). In the outlier region 90, the distance from the center of the distribution is large and thus the value of the distance z_score is large. Thus, regarding the data in the outlier region 90, the value of the power expressed by Expression (2), namely, the weight is large and thus the loss L_{org1} is large. Therefore, the influence of the data in the outlier region on the loss L_{org1} can be increased.

A probability distribution of training data is not necessarily accurately a normal distribution, and thus a skewness k may be taken into account, depending on a probability distribution of training data to be used. In this case, the skewness k is taken into account in the loss function L_{org1} for the probability distribution of training data, that is, a loss function L_{org2} in Expression (3) is used. The skewness k is calculated, based on the probability distribution.

$${L}_{org 2} = {\vert y - f ( x ) \vert}^{a * ( 1 - {e}^{\frac{- b \vert z \_ score \vert}{k}} ) + c} \qquad ( 3 )$$

$$k : SKEWNESS$$

FIG. 3 is an explanatory graph for the hyperparameters a, b, and c in Expressions (1) to (3). In FIG. 3, the horizontal axis represents the distance z_score from the center of the probability distribution, and the vertical axis represents the power. The hyperparameter a controls the maximum value of the power. The hyperparameter b controls the rising rate of the power. The hyperparameter c indicates the power in a case where the distance z_score is 0, namely, a starting point for the power. The hyperparameters a, b, and c are basically set by a user.

### First Example Embodiment

### (Entire Configuration)

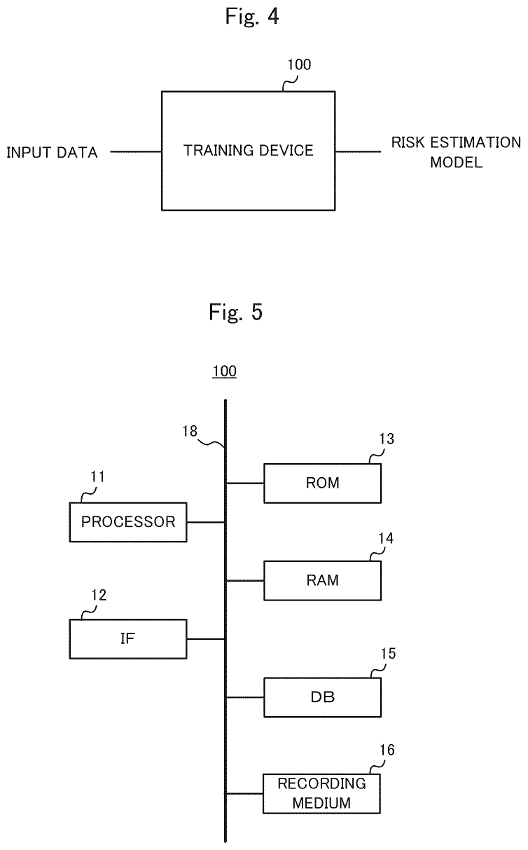

FIG. 4 illustrates the entire configuration of a training device according to the present disclosure. A training device 100 trains a risk estimation model for estimating a disease risk to a subject, based on data regarding the health of the subject. Specifically, the training device 100 receives input data regarding the health of the subject. The input data includes various types of data obtained in a medical examination, such as the height, weight, sex, blood pressure, body mass index (BMI), body fat percentage, neutral fat level, smoking status or amount, and drinking status or amount of the subject. Note that the input data may be multimodal data, namely, a plurality of pieces of data in the above-described various types of data.

The risk estimation model generated by the training device 100 can be favorably applied to the field of medicine or healthcare. For example, the risk estimation model can be used to estimate the risk of any lifestyle-related disease, based on data obtained in a regular medical examination.

### (Hardware Configuration)

FIG. 5 is a block diagram illustrating a hardware configuration of the training device 100. As illustrated, the training device 100 includes a processor 11, an interface (IF) 12, a read only memory (ROM) 13, a random access memory (RAM) 14, a database (DB) 15, and a recording medium 16. For example, the constituent elements are mutually connected through a bus 18.

The processor 11 corresponds to a computer, such as a central processing unit (CPU), and executes a program prepared in advance to control the entire training device 100. Specifically, as the processor 11, a CPU, a graphics processing unit (GPU), a digital signal processor (DSP), a micro processing unit (MPU), a floating point number processing unit (FPU), a physics processing unit (PPU), a tensor processing unit (TPU), a quantum processor, a microcontroller, or any combination thereof can be used.

In addition, the processor 11 loads a program stored in the ROM 13 or the recording medium 16 into the RAM 14 and performs each piece of processing coded in the program. The processor 11 functions as part of the training device 100 or the entirety of the training device 100. Specifically, the processor 11 performs training processing, which will be described later.

The IF 12 transmits/receives data to/from an external device. Specifically, the training device 100 receives, as training data, input data regarding the health of a plurality of persons through the IF 12. In addition, the training device 100 outputs a trained risk estimation model to a storage device or an external device through the IF 12.

The ROM 13 stores various types of programs that the processor 11 executes. The RAM 14 is used as a working memory while the processor 11 is performing various types of processing.

The DB 15 stores various types of algorithms, data, and machine learning models for use when the training device 100 performs the training processing, which will be described later.

The recording medium 16 corresponds to a non-volatile and non-transitory storage medium, such as a disk-shaped recording medium or a semiconductor memory. The recording medium 16 may be detachably attached to the training device 100. The recording medium 16 has various types of programs, which the processor 11 executes, recorded thereon.

Note that, in addition to the above, the training device 100 may include a display device, such as a liquid crystal display, and an input device, such as a keyboard or a mouse. Such a display device and an input device are used, for example, by an operator for the training device 100.

### (Functional Configuration)

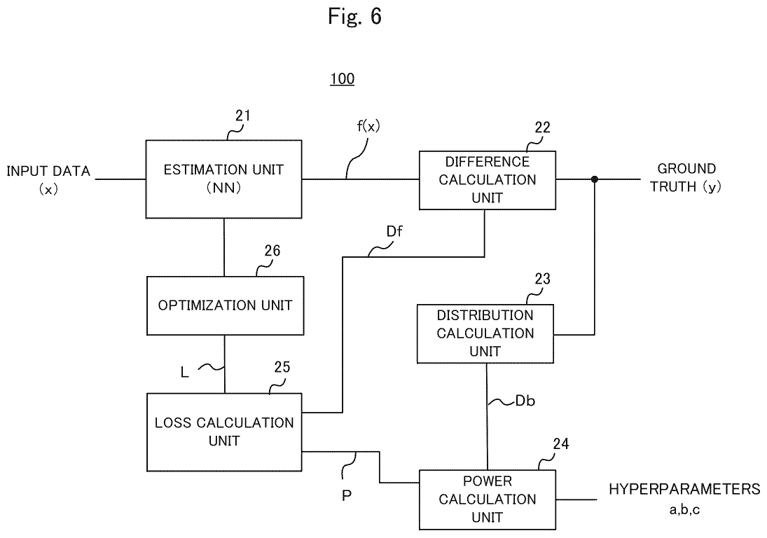

FIG. 6 is a block diagram illustrating a functional configuration of the training device 100. The training device 100 includes an estimation unit 21, a difference calculation unit 22, a distribution calculation unit 23, a power calculation unit 24, a loss calculation unit 25, and an optimization unit 26.

The estimation unit 21 includes a neural network (NN) that functions as a risk estimation model. Input data x included in a data set of training data is input to the estimation unit 21. The input data x corresponds to an explanatory variable. The estimation unit 21 estimates a risk from the input data x using the risk estimation model and outputs an estimated value f(x) to the difference calculation unit 22. The difference calculation unit 22 calculates a difference Df between the estimated value f(x) and a ground truth y and outputs the difference Df to the loss calculation unit 25. Note that the ground truth y corresponds to a response variable (objective variable) to the explanatory variable x.

The distribution calculation unit 23 calculates a probability density function of the ground truth y of the training data to obtain a probability distribution. The distribution calculation unit 23 outputs, to the power calculation unit 24 and the loss calculation unit 25, a parameter Db that defines the obtained probability distribution. As an example, the parameter Db includes the average u and the standard deviation o.

The hyperparameters a, b, and c are set to the power calculation unit 24. The power calculation unit 24 obtains the distance z_score from the parameter Db, calculates a power P based on Expression (2), and outputs the power P to the loss calculation unit 25.

Based on the difference Df input from the difference calculation unit 22 and the power P input from the power calculation unit 24, the loss calculation unit 25 calculates a loss L (L_{org1} or L_{org2}) using the loss function expressed by Expression (1) or the loss function expressed by Expression (3) and outputs the loss L to the optimization unit 26.

The optimization unit 26 optimizes a parameter of the neural network included in the estimation unit 21 in such a way that the loss L input from the loss calculation unit 25 is minimized. In this manner, the risk estimation model, which includes the neural network, is trained.

In the above-described configuration, the estimation unit 21 is an example of an estimation means, the difference calculation unit 22 is an example of a difference calculation means, and the distribution calculation unit 23 is an example of a distribution calculation means. In addition, the loss calculation unit 25 is an example of a loss calculation means, and the optimization unit 26 is an example of a training means.

### (Training Processing)

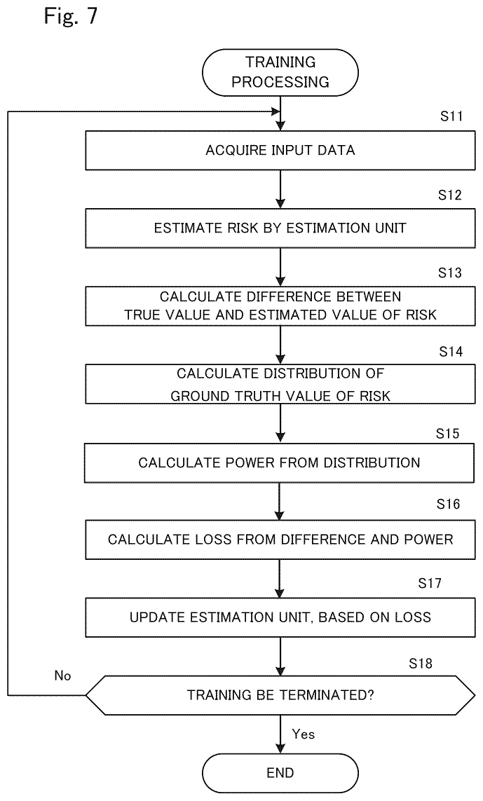

Next, the training processing that the above-described training device 100 performs will be described. FIG. 7 is a flowchart of the training processing. The processor 11 illustrated in FIG. 5 executes the program prepared in advance to operate as the constituent elements illustrated in FIG. 6, so that the processing is achieved.

First, the estimation unit 21 acquires input data included in training data (step S11). Next, the estimation unit 21 estimates a risk from the input data using the risk estimation model formed by the neural network (step S12). Next, the difference calculation unit 22 calculates the difference between the ground truth and the estimated value of the risk (step S13). Next, the distribution calculation unit 23 calculates a probability distribution of the ground truth of the risk (step S14). Next, the power calculation unit 24 calculates a power from the probability distribution of the ground truth (step S15).

Next, the loss calculation unit 25 calculates a loss from the difference and the power (step S16). Next, the optimization unit 26 updates the parameter of the neural network of the estimation unit 21 in such a way that the loss is reduced (step S17).

Next, the training device 100 determines whether a predetermined training termination condition is fulfilled (step S18). Examples of the training termination condition include a condition that a predetermined number of pieces of data prepared as the training data have been used, a condition that the loss L is equal to or less than a predetermined value, and a condition that the loss L has converged. In a case where the training termination condition is not fulfilled (step S18: No), the processing goes back to step S11. On the other hand, in a case where the training termination condition is fulfilled (step S18: Yes), the training processing terminates.

### (Experimental Results)

An estimation model for estimating a change in blood glucose level in the future was trained based on data of the blood glucose level obtained in a medical examination. At this time, as loss functions, L1 to L4 norms, the Huber loss, the Cauchy loss, the Welsch loss, the Elastic loss, the Quantile loss, and the losses L_{org1} and L_{org2} of the proposed technique were used. Using a difference in age, in addition to a difference in blood glucose level, as explanatory variables, a model for estimating a change in blood glucose level in N years was generated.

As evaluation indicators, an intraclass correlation coefficient (ICC) and mean absolute error (MAE) accuracy were used. According to the models using the loss functions of the proposed technique, an improvement was observed in the intraclass correlation coefficient, and an improvement in MAE accuracy was observed regarding the data in a range from 1.5σ and above associated to the outlier region in a probability distribution of training data. On the other hand, regarding most of the data in the probability distribution (data in a range from −1.5σ to 1.5σ), the intraclass correlation coefficient and the MAE accuracy were maintained at a similar level to those using the other loss functions. Thus, due to the loss functions L_{org1} and L_{org2} of the proposed technique, an improvement in accuracy to the data corresponding to outliers was observed with the performance retained to most of the data in a distribution of training data.

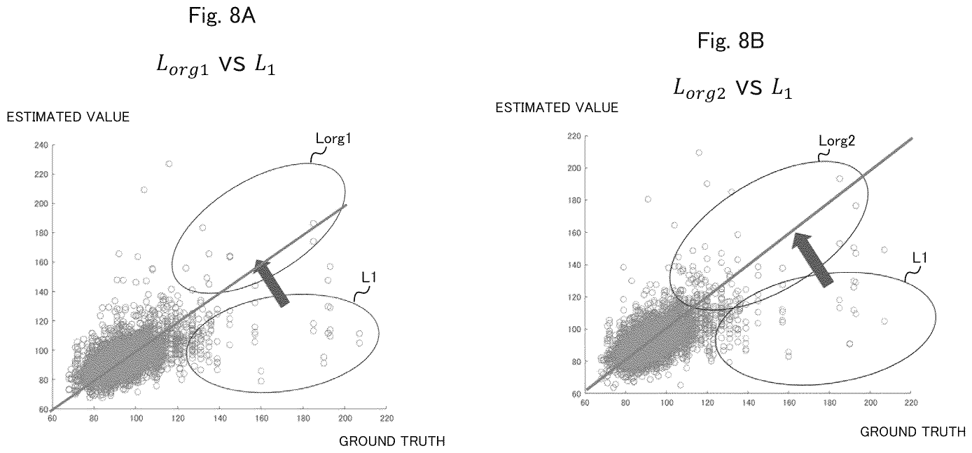

In addition, cases using the loss functions L_{org1} and L_{org2} of the proposed technique and a case using the L1 loss were compared in the degree of agreement between an estimated value from the risk estimation model obtained by training and a ground truth. FIGS. 8A and 8B are graphs in which the ground truth and the estimated value are plotted. FIG. 8A is a graph of comparison between the loss L_{org1} of the proposed technique and the L1 loss, and FIG. 8B is a graph of comparison between the loss L_{org2} of the proposed technique and the L1 loss. In each graph, the horizontal axis represents the ground truth and the vertical axis represents the estimated value. A line segment in each drawing indicates a perfect agreement line. Referring to FIGS. 8A and 8B, the regions of location of ellipses L_{org1} and L_{org2} indicating plots using the loss functions of the proposed technique are each closer to the perfect agreement line than the region of location of an ellipse L1 using the L1 loss is, indicating a higher accuracy of the estimated value. That is, it is found that use of the loss functions of the proposed technique enables a high estimation accuracy even to data deviating from the center of a distribution of training data, in comparison to use of the L1 loss.

### Application Examples

Application examples of the above-described example embodiment will be described below.

### First Application Example

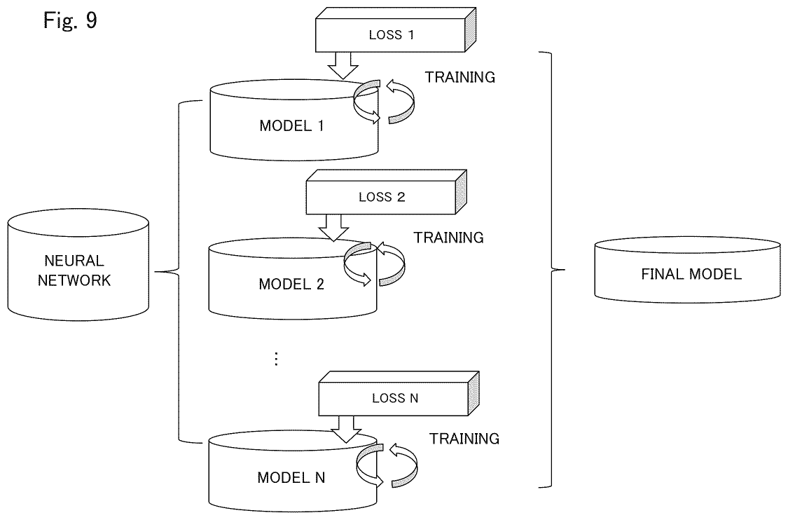

FIG. 9 illustrates an application example of the training device according to the example embodiment. A model is trained using a plurality of different loss functions including the loss functions of the proposed technique to generate a plurality of models different in characteristic. Then, a final model is generated by ensemble training using the generated plurality of models. Thus, a final model adaptive to various characteristics of input data can be constructed.

### Second Application Example

As described above, the loss functions of the proposed technique can be applied to training of a risk estimation model for estimating a disease risk based on medical examination data. In this case, the “difference in age” is added together with particular medical examination data (e.g., the blood glucose level) to input data corresponding to the explanatory variable, enabling estimation of a disease risk in N years.

The difference in age is set at 0 in the risk estimation model, enabling estimation of another disease risk at the corresponding age of a subject. For example, in a case where a particular item of data is lacking in the medical examination data at a certain age of a certain subject, the particular item of data can be interpolated. In addition, based on the attribute of a subject, data of a virtual person similar in attribute to the subject can be generated. In addition, for a subject whose data at a certain age is missing, the missing data at the certain age can be generated using the data at the certain age of another subject.

For example, in a case where ten years of medical examination data of a plurality of persons is accumulated by collection in a continuous medical examination, the data is divided every two years to create groups G1 to G5 of data. Then, a difference is obtained based on any appropriate combination of the groups G1 to G5 of data, and medical examination data associated to a desired difference in age for the same person is created for use in training.

Furthermore, the groups G1 to G5 of data are classified based on the age of a subject, so that medical examination data associated to each age group can be created. Thus, from ten years of data of persons in each age group, medical examination data in an age range of 10 years or more can be created in a pseudo-manner, and thus a risk estimation model capable of estimating a longer-term disease risk can be constructed.

### Second Example Embodiment

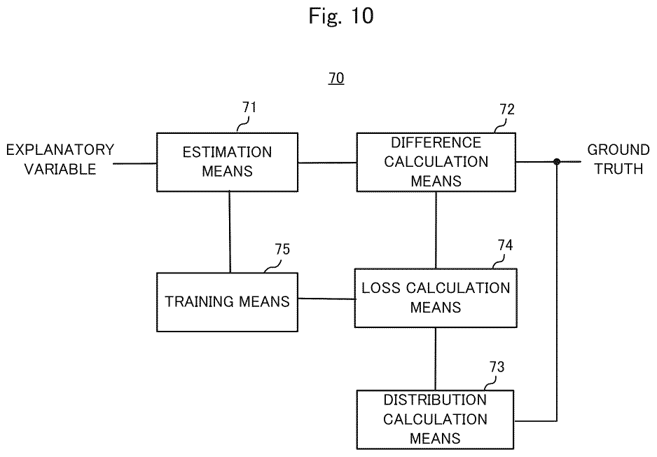

FIG. 10 is a block diagram illustrating a functional configuration of a training device according to a second example embodiment. A training device 70 includes an estimation means 71, a difference calculation means 72, a distribution calculation means 73, a loss calculation means 74, and a training means 75.

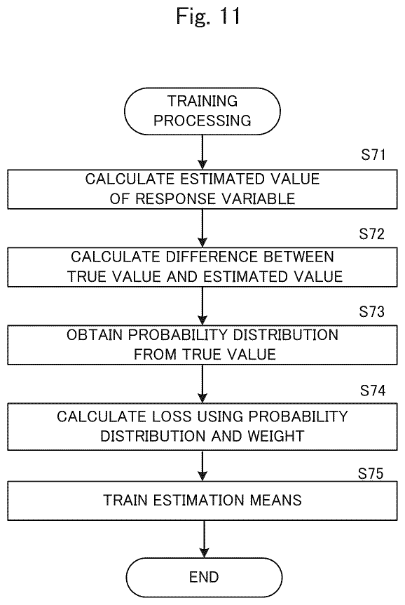

FIG. 11 is a flowchart of processing that the training device according to the second example embodiment performs. The estimation means 71 calculates an estimated value of a response variable from an input explanatory variable (step S71). The difference calculation means 72 calculates the difference between a ground truth of the response variable and the estimated value (step S72). The distribution calculation means 73 calculates a probability density function from the ground truth of the response variable to obtain a probability distribution (step S73). The loss calculation means 74 calculates a loss by weighting the difference by a weight based on the probability distribution (step S74). The training means 75 trains the estimation means 71, based on the loss (step S75).

The training device 70 according to the second example embodiment enables training with effective use of data deviating from the center in a distribution of training data.

A part or all of the example embodiments described above may also be described as the following supplementary notes, but not limited thereto.

### (Supplementary Note 1)

A training device comprising:
an estimation means for calculating an estimated value of a response variable from an input explanatory variable using an estimation model;
a difference calculation means for calculating a difference between a ground truth of the response variable and the estimated value;
a distribution calculation means for calculating a probability density function from the ground truth of the response variable to obtain a probability distribution;
a loss calculation means for calculating a loss by weighting the difference by a weight based on the probability distribution; and
a training means for training the estimation model, based on the loss.

### (Supplementary Note 2)

The training device according to Supplementary note 1, wherein the loss calculation means assigns a larger weight to the difference associated with the ground truth located further away from a center in the probability distribution.

### (Supplementary Note 3)

The training device according to Supplementary note 1, wherein the loss calculation means calculates a power in a Minkowski distance based on a parameter of the probability density function, and calculates the loss based on the difference and the power.

### (Supplementary Note 4)

The training device according to Supplementary note 3, wherein the parameter is a distance of the ground truth to a center of the probability distribution.

### (Supplementary Note 5)

The training device according to Supplementary note 3, wherein the loss calculation means calculates the loss using a loss function in which the difference is exponentiated by an exponent indicated by the power.

### (Supplementary Note 6)

The training device according to Supplementary note 3, wherein the power is calculated using a first hyperparameter indicating a maximum value of the power, a second hyperparameter indicating a rising rate of the power, and a third hyperparameter indicating a value of the power at a center of the probability distribution.

### (Supplementary Note 7)

The training device according to Supplementary note 3, wherein the power is calculated using a skewness of the probability distribution.

### (Supplementary Note 8)

The training device according to Supplementary note 1, wherein the estimation model is a deep learning model, wherein the explanatory variable is data regarding health of a person, and wherein the response variable is a disease risk to the person.

### (Supplementary Note 9)

A training method to be performed by a computer, comprising:
calculating an estimated value of a response variable from an input explanatory variable using an estimation model;
calculating a difference between a ground truth of the response variable and the estimated value;
calculating a probability density function from the ground truth of the response variable to obtain a probability distribution;
calculating a loss by weighting the difference by a weight based on the probability distribution; and
training the estimation model, based on the loss.

### (Supplementary Note 10)

A non-transitory computer-readable recording medium storing a program, the program causing a computer to perform processing comprising:
calculating an estimated value of a response variable from an input explanatory variable using an estimation model;
calculating a difference between a ground truth of the response variable and the estimated value;
calculating a probability density function from the ground truth of the response variable to obtain a probability distribution;
calculating a loss by weighting the difference by a weight based on the probability distribution; and
training the estimation model, based on the loss.

Some or all of the configurations described in Supplementary Notes 2 to 8 dependent on the above-described Supplementary Note 1 may also be dependent on Supplementary Notes 9 and 10 due to a dependency relationship similar to that of Supplementary Notes 2 to 8. Furthermore, some or all of the configurations described as Supplementary Notes may be dependent on not only Supplementary Notes 1, 9, and 10, but also various types of hardware and software, various recording means for recording software, and systems without departing from the above-described example embodiments.

While the present disclosure has been particularly shown and described with reference to example embodiments and examples thereof, the present disclosure is not limited to these example embodiments and examples. It will be understood by those of ordinary skill in the art that various changes in form and details may be made therein without departing from the spirit and scope of the present disclosure as defined by the claims.

### DESCRIPTION OF SYMBOLS

11 Processor
21 Estimation unit
22 Difference calculation unit
23 Distribution calculation unit
24 Power calculation unit
25 Loss calculation unit
26 Optimization unit
100 Training device

1. A training device comprising:
an interface configured to transmit and receive data;
a memory configured to store instructions and training data; and
a processor configured to execute the instructions to:
acquire, via the interface, the training data including (i) medical examination data obtained in a regular medical examination of a person as an explanatory variable, and (ii) a ground truth of a response variable indicating at least one of a change in a blood glucose level in N years and a disease risk in N years;
calculate an estimated value of a response variable from an input explanatory variable using an estimation model;
calculate a difference between a ground truth of the response variable and the estimated value;
calculate a probability density function from the ground truth of the response variable to obtain a probability distribution;
calculate a weight based on the probability distribution and a loss by weighting the difference by a weight based on the probability distribution; and
train the estimation model, based on the loss, to generate a risk estimation model used to estimate the response variable based on data obtained in the regular medical examination; and output the risk estimation model via the interface.

2. The training device according to claim 1, wherein the processor assigns a larger weight to the difference associated with the ground truth located further away from a center in the probability distribution.

3. The training device according to claim 1, wherein the processor calculates a power in a Minkowski distance based on a parameter of the probability density function, and calculates the loss based on the difference and the power.

4. The training device according to claim 3, wherein the parameter is a distance of the ground truth to a center of the probability distribution.

5. The training device according to claim 3, wherein the processor calculates the loss using a loss function in which the difference is exponentiated by an exponent indicated by the power.

6. The training device according to claim 3, wherein the power is calculated using a first hyperparameter indicating a maximum value of the power, a second hyperparameter indicating a rising rate of the power, and a third hyperparameter indicating a value of the power at a center of the probability distribution.

7. The training device according to claim 3, wherein the power is calculated using a skewness of the probability distribution.

8. The training device according to claim 1,
wherein the estimation model is a deep learning model or a machine learning model,
wherein the explanatory variable is the medical examination data obtained in the regular medical examination of the person, and
wherein the response variable is the disease risk in N years to the person.

9. A training method performed by a computer, comprising:
acquiring, via an interface, training data including (i) medical examination data obtained in a regular medical examination of a person as an explanatory variable, and (ii) a ground truth of a response variable indicating at least one of a change in a blood glucose level in N years and a disease risk in N years;
calculating an estimated value of the response variable from an input of an explanatory variable by using an estimation model;
calculating a difference between a ground truth of the response variable and the estimated value;
calculating a probability density function from the ground truth of the response variable to obtain a probability distribution;
calculating a weight based on the probability distribution and calculating a loss by weighting the difference by the weight; and
training the estimation model, based on the loss, to generate a risk estimation model used to estimate the response variable based on data obtained in the regular medical examination; and outputting the risk estimation model via the interface.

10. A non-transitory computer-readable recording medium storing instructions, the instructions causing a computer to perform processing comprising:
acquiring, via an interface, training data including (i) medical examination data obtained in a regular medical examination of a person as an explanatory variable, and (ii) a ground truth of a response variable indicating at least one of a change in a blood glucose level in N years and a disease risk in N years;
calculating an estimated value of the response variable from an input of the explanatory variable by using an estimation model;
calculating a difference between a ground truth of the response variable and the estimated value;
calculating a probability density function from the ground truth of the response variable to obtain a probability distribution;
calculating a weight based on the probability distribution and calculating a loss by weighting the difference by the weight; and
training the estimation model, based on the loss, to generate a risk estimation model used to estimate the response variable based on data obtained in the regular medical examination; and outputting the risk estimation model via the interface.
